(19) United States
(12) Patent Application Publication
(10) Pub. No.: US 2018/0325850 A1
(43) Pub. Date: Nov. 15, 2018

(54) VALPROIC ACID FOR TREATMENT OF MYOCARDIAL ISCHEMIA

(71) Applicant: The Regents of the University of Michigan, Ann Arbor, MI (US)

(72) Inventors: Zhong Wang, Ann Arbor, MI (US); Francis D. Pagani, Ann Arbor, MI (US); Yuqing Chen, Ann Arbor, MI (US); Hasan Alam, Ann Arbor, MI (US); Yongqing Li, Ann Arbor, MI (US)

(21) Appl. No.: 15/977,700

(22) Filed: May 11, 2018

Related U.S. Application Data
(60) Provisional application No. 62/505,272, filed on May 12, 2017.

(51) Int. Cl. A61K 31/19; A61K 9/00; A61K 45/06; A61K 31/616; A61K 31/4365; A61K 31/04; A61P 9/10

(52) U.S. Cl. CPC A61K 31/19; A61K 9/0053; A61K 9/0019; A61P 9/10; A61K 31/616; A61K 31/4365; A61K 31/04; A61K 45/06

(57) ABSTRACT

Provided herein are pharmaceutical compositions comprising valproic acid and methods of use thereof for the treatment and/or prevention of myocardial ischemia and/or infarction.

### CROSS-REFERENCE TO RELATED APPLICATION

The present application claims priority to U.S. Provisional Patent Application Ser. No. 62/505,272 filed May 12, 2017, which is hereby incorporated by reference in its entirety.

### FIELD

Provided herein are pharmaceutical compositions comprising valproic acid (VPA) and methods of use thereof for the treatment and/or prevention of myocardial ischemia and/or infarction (MI).

### BACKGROUND

Cardiovascular disease (CVD) is the leading cause of death in the world and USA today (American Heart Association, AHA). In particular, myocardial infarction, commonly known as heart attack, results in permanent heart muscle damage or death, and is the number one killer of heart patients. Every year about 660,000 Americans have a new heart attack and about 305,000 American have a recurrent attack. Among these patients, 370,000 die from the disease. Patients who survive the heart attack often develop heart failure and sudden cardiac arrest due to severely weakened pumping function of the heart. A consequence of heart attack is the heart muscle cell loss. A typical human heart attack causes the loss of approximately 1 billion heart muscle cells. These statistics underscore the critical need for developing effective therapeutic strategies to prevent MI and/or preserve the pumping function after infarction and/or ischemia.

Valproic acid (VPA) is an FDA approved drug for treating epilepsy and bipolar disorder and to prevent migraine headaches.

### SUMMARY

Provided herein are pharmaceutical compositions comprising valproic acid and methods of use thereof for the treatment and/or prevention of myocardial ischemia and/or infarction.

In some embodiments, provided herein are methods of reducing the risk of myocardial infarction (MI) in a subject comprising administering to the subject an effective dose of valproic acid or a related form thereof. In some embodiments, the subject does not suffer from one or more (all) of epilepsy, mania, bipolar disorder, migrane headaches, or a seizure disorder. In some embodiments, the subject is at increased risk of MI relative to the general population. In some embodiments, the subject suffers from myocardial ischemia, congenital heart disease, and/or atherosclerosis. In some embodiments, the subject is administered a related from of valproic acid selected from the group consisting of valproate, sodium valproate, and divalproex. In some embodiments, valproic acid or related form thereof is administered orally or by injection. In some embodiments, valproic acid or related form thereof is co-administered with one or more additional prophylactic agents. In some embodiments, the one or more additional prophylactic agents are selected from the group consisting of aspirin, a statin, clopidogrel, an anticoagulant, a beta-blocker, and an ACE inhibitor. In some embodiments, the valproic acid or related form thereof and the one or more additional prophylactic agents are co-formulated. In some embodiments, the valproic acid or a related form thereof and the one or more additional prophylactic agents are contained in separate pharmaceutical compositions.

In some embodiments, provided herein are prophylactic pharmaceutical compositions for the prevention of myocardial infarction comprising co-formulated: (i) valproic acid or a related form thereof, and (ii) aspirin, a statin, clopidogrel, an anticoagulant, a beta-blocker, and an ACE inhibitor.

In some embodiments, provided herein are kits for the prophylactic prevention of myocardial infarction comprising separately-formulated: (i) valproic acid or a related form thereof, and (ii) aspirin, a statin, clopidogrel, an anticoagulant, a beta-blocker, and an ACE inhibitor.

In some embodiments, provided herein are methods of reducing tissue damage caused by myocardial infarction (MI) in a subject comprising administering to the subject an effective dose of valproic acid or a related form thereof. In some embodiments, the subject does not suffer from one or more (all) of epilepsy, mania, bipolar disorder, migrane headaches, or a seizure disorder. In some embodiments, the administration of valproic acid or a related form thereof reduces tissue damage caused by the MI event itself. In some embodiments, the administration of valproic acid or a related form thereof reduces tissue damage caused by reperfusion. In some embodiments, the subject is at increased risk of MI relative to the general population and is administered valproic acid or a related form thereof before an MI event occurs. In some embodiments, the subject suffers from myocardial ischemia, congenital heart disease, and/or atherosclerosis. In some embodiments, the subject is administered valproic acid or a related form thereof during an MI event. In some embodiments, the subject is administered valproic acid or a related form thereof after an MI event. In some embodiments, the subject is co-administered one or more additional prophylactic agents selected from the group consisting of aspirin, a thrombolytic agent, an antiplatelet agent, a blood thinner, a pain reliever, nitroglycerin, a beta blocker, and an ACE inhibitor. In some embodiments, the subject is administered a related from of valproic acid selected from the group consisting of valproate, sodium valproate, and divalproex. In some embodiments, the valproic acid or related form thereof is administered by injection. In some embodiments, the valproic acid or related form thereof is injected intraperitoneally.

In some embodiments, provided herein are pharmaceutical compositions for treatment and/or reduction of tissue damage from myocardial infarction comprising co-formulated: (i) valproic acid or a related form thereof, and (ii) aspirin, a thrombolytic agent, an antiplatelet agent, a blood thinner, a pain reliever, nitroglycerin, a beta blocker, and an ACE inhibitor. In some embodiments, provided herein are for the treatment of myocardial infarction and/or reduction of tissue damage from myocardial infarction comprising separately-formulated: (i) valproic acid or a related form thereof, and (ii) aspirin, a statin, clopidogrel, an anticoagulant, a beta-blocker, and an ACE inhibitor.

In some embodiments, provided herein is the use of a pharmaceutical composition or kit described herein for the treatment, prevention, or reduction of symptoms resulting from myocardial infarction.

In some embodiments, provided herein is the use of a pharmaceutical composition or kit described herein in the manufacture of a medicament for the treatment, prevention, or reduction of symptoms resulting from myocardial infarction.

### BRIEF DESCRIPTION OF THE DRAWINGS

FIG. 1. VPA treatment within 12 h after MI preserved heart function. VPA treatment significantly improved ejection fraction (EF) (Panel A) and fractional shortening (FS) (Panel B). VPA treatment rescued roughly 50% of the functional loss caused by acute IR injury.

FIG. 2. VPA treatment within 12 h after MI reduced infarct size as indicated by triphenyltetrazolium chloride staining (Panel A) and statistical analysis (Panel B). VPA treatment reduced infarct size by around 45%.

FIG. 3. VPA reduced MI injury as measured by plasma biochemical assays. (Panel A) Reduction of Lactate dehydrogenase (LDH). (Panel B) Reduction of creatine kinase (CK).

FIG. 4. VPA targets identified by RNA-seq.

FIG. 5. A diagram for VPA mediated gene expression and heart regeneration.

### DEFINITIONS

Although any methods and materials similar or equivalent to those described herein can be used in the practice or testing of embodiments described herein, some preferred methods, compositions, devices, and materials are described herein. However, before the present materials and methods are described, it is to be understood that this invention is not limited to the particular molecules, compositions, methodologies or protocols herein described, as these may vary in accordance with routine experimentation and optimization. It is also to be understood that the terminology used in the description is for the purpose of describing the particular versions or embodiments only, and is not intended to limit the scope of the embodiments described herein.

Unless otherwise defined, all technical and scientific terms used herein have the same meaning as commonly understood by one of ordinary skill in the art to which this invention belongs. However, in case of conflict, the present specification, including definitions, will control. Accordingly, in the context of the embodiments described herein, the following definitions apply.

As used herein and in the appended claims, the singular forms “a”, “an” and “the” include plural reference unless the context clearly dictates otherwise. Thus, for example, reference to “a therapeutic agent” is a reference to one or more therapeutic agents and equivalents thereof known to those skilled in the art, and so forth.

As used herein, the term “comprise” and linguistic variations thereof denote the presence of recited feature(s), element(s), method step(s), etc. without the exclusion of the presence of additional feature(s), element(s), method step(s), etc. Conversely, the term “consisting of” and linguistic variations thereof, denotes the presence of recited feature(s), element(s), method step(s), etc. and excludes any unrecited feature(s), element(s), method step(s), etc., except for ordinarily-associated impurities. The phrase “consisting essentially of” denotes the recited feature(s), element(s), method step(s), etc. and any additional feature(s), element(s), method step(s), etc. that do not materially affect the basic nature of the composition, system, or method. Many embodiments herein are described using open “comprising” language. Such embodiments encompass multiple closed “consisting of” and/or “consisting essentially of” embodiments, which may alternatively be claimed or described using such language.

As used herein, the term “valproic acid” refers to a molecule with the chemical structure of:

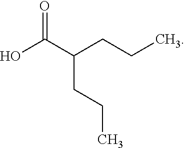

As used herein, the term “related forms,” when used in reference to valproic acid, refers to:

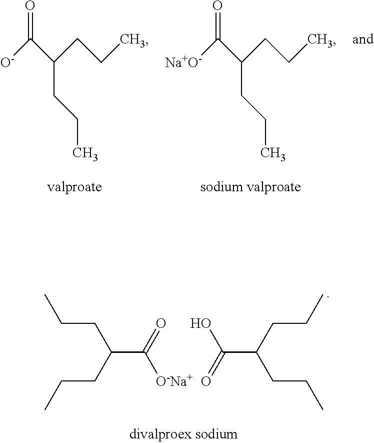

As used herein, the term “myocardial ischemia” refers to circulatory disturbances caused by coronary atherosclerosis and resulting in reduced blood, oxygen and/or nutrient supply to the myocardium.

The term “myocardial infarction” (MI) (a.k.a. “heart attack”) refers to a severe ischemic insult to myocardial tissue resulting from a lack of blood flow to the affected area. Specifically, this insult results from an occlusive (e.g., thrombotic or embolic) event in the coronary circulation that stops blood flow to a region of the heart muscle and produces an environment in which the myocardial metabolic demands exceed the supply of oxygen and or nutrients.

As used herein, the term “subject” broadly refers to any animal, including but not limited to, human and non-human animals (e.g., dogs, cats, cows, horses, sheep, poultry, fish, crustaceans, etc.). As used herein, the term “patient” typically refers to a subject that is being treated for a disease or condition.

As used herein, the term “pharmaceutical composition” refers to the combination of an active agent with a carrier, inert or active, making the composition especially suitable for diagnostic or therapeutic use in vitro, in vivo or ex vivo.

The terms “pharmaceutically acceptable” or “pharmacologically acceptable,” as used herein, refer to compositions that do not substantially produce adverse reactions, e.g., toxic, allergic, or immunological reactions, when administered to a subject.

As used herein, the term “pharmaceutically acceptable carrier” refers to any of the standard pharmaceutical carriers including, but not limited to, phosphate buffered saline solution, water, emulsions (e.g., such as an oil/water or water/oil emulsions), and various types of wetting agents, any and all solvents, dispersion media, coatings, sodium lauryl sulfate, isotonic and absorption delaying agents, disintigrants (e.g., potato starch or sodium starch glycolate), and the like. The compositions also can include stabilizers and preservatives. For examples of carriers, stabilizers and adjuvants, see, e.g., Martin, Remington's Pharmaceutical Sciences, 15th Ed., Mack Publ. Co., Easton, Pa. (1975), incorporated herein by reference in its entirety.

As used herein, the terms “administration” and “administering” refer to the act of giving a drug, prodrug, or other agent, or therapeutic treatment to a subject or in vivo, in vitro, or ex vivo cells, tissues, and organs. Exemplary routes of administration to the human body can be through space under the arachnoid membrane of the brain or spinal cord (intrathecal), the eyes (ophthalmic), mouth (oral), skin (topical or transdermal), nose (nasal), lungs (inhalant), oral mucosa (buccal), ear, rectal, vaginal, by injection (e.g., intravenously, subcutaneously, intratumorally, intraperitoneally, etc.) and the like.

As used herein, the terms “co-administration” and “co-administering” refer to the administration of at least two agent(s) (e.g., VPA or a related form thereof and a second agent) or therapies to a subject. In some embodiments, the co-administration of two or more agents or therapies is concurrent. In other embodiments, a first agent/therapy is administered prior to a second agent/therapy. Those of skill in the art understand that the formulations and/or routes of administration of the various agents or therapies used may vary. The appropriate dosage for co-administration can be readily determined by one skilled in the art. In some embodiments, when agents or therapies are co-administered, the respective agents or therapies are administered at lower dosages than appropriate for their administration alone. Thus, co-administration is especially desirable in embodiments where the co-administration of the agents or therapies lowers the requisite dosage of a potentially harmful (e.g., toxic) agent(s), and/or when co-administration of two or more agents results in sensitization of a subject to beneficial effects of one of the agents via co-administration of the other agent.

As used herein, the term “effective amount” refers to the amount of a composition sufficient to effect beneficial or desired results. An effective amount can be administered in one or more administrations, applications or dosages and is not intended to be limited to a particular formulation or administration route.

As used herein, the term “treating” refers to inhibiting a disease, disorder or condition (e.g., myocardial ischemia) in a subject. Treating the disease or condition includes ameliorating at least one symptom, reducing severity, impeding progress, and/or curing the subject of the disease or condition.

As used herein, the term “preventing” refers to prophylactic steps taken to reduce the likelihood of a subject (e.g., an at-risk subject) from contracting or suffering from a particular disease, disorder or condition (e.g., myocardial infarction). The likelihood of the disease, disorder or condition occurring in the subject need not be reduced to zero for the preventing to occur; rather, if the steps reduce the risk of a disease, disorder or condition across a population, then the steps prevent the disease, disorder or condition within the scope and meaning herein.

### DETAILED DESCRIPTION

Provided herein are pharmaceutical compositions comprising valproic acid and methods of use thereof for the treatment and/or prevention of myocardial ischemia and/or infarction.

Valproic acid (VPA), and its valproate, sodium valproate, and divalproex sodium forms are histone deacetylases (HDAC) inhitors. HDAC inhibitors, by definition, are chemical compounds that inhibit histone deacetylases (HDACs) (Refs. 6-8; incorporated by reference in their entireties). In general, HDACs remove the acetyl groups from the lysine residues and render chromatin more condensed and transcriptionally silenced. HDAC inhibitors prevent this action and affect gene expression. Valproic acid and other HDAC inhibitors have been used as mood stabilizers and anti-epileptics. More recent advances demonstrate utility in treating cancers, parasitic, and inflammatory diseases (Refs. 9-12; incorporated by reference in their entireties). VPA is approved drug primarily for epilepsy and bipolar disorders. It is one of the most important medications in a basic health system and on the World Health Organization's list of Essential Medicines (Ref. 16; incorporated by reference in its entirety).

Experiments conducted during development of embodiments herein demonstrate, for example, that VPA significantly reduces infarct size when administrated at the onset of ischemia reperfusion (IR), and that VPA preserves the pumping function of heart after IR in a rat model of MI, in both short term and long term. Therefore, in some embodiments, provided herein are methods of reducing the risk of MI in a subject by the administration of VPA, as described in more detail herein. Also, in some embodiments, provided herein are methods of reducing the damage (e.g., tissue damage) resulting from MI or reperfusion thereafter in a subject by the administration of VPA, as described in more detail herein.

Pharmaceutical compositions include compositions wherein the active ingredients (e.g., VPA) are contained in an effective amount to achieve the intended purpose. For example, an effective amount of VPA may be that amount that reduces the risk of MI and/or reduces tissue damage from MI. Determination of effective amounts is well within the capability of those skilled in the art, especially in light of the disclosure provided herein.

In addition to the active ingredients (e.g., VPA), pharmaceutical compositions may contain suitable pharmaceutically acceptable carriers comprising excipients and auxiliaries that facilitate processing of the active compounds into preparations that can be used pharmaceutically. The preparations formulated for oral administration may be in the form of tablets, dragees, capsules, or solutions. The pharmaceutical compositions of the present invention may be manufactured in a manner that is itself known (e.g., by means of conventional mixing, dissolving, granulating, dragee-making, levigating, emulsifying, encapsulating, entrapping or lyophilizing processes).

Certain embodiments herein provide pharmaceutical compositions that may be administered by any suitable route, such as pulmonary (e.g., by inhalation or insufflation of powders or aerosols, including by nebulizer; intratracheal, intranasal, epidermal and transdermal), oral, or parenteral. Parenteral administration includes intravenous, intraarterial, subcutaneous, intraperitoneal or intramuscular injection or infusion; or intracranial, e.g., intrathecal or intraventricular, administration. In certain embodiments, intraperitoneal administration is used.

Pharmaceutical formulations for parenteral administration include aqueous solutions of the active compounds in water-soluble form. Compositions and formulations for parenteral administration (e.g., intraperitoneal, intravenous, etc.) may include sterile aqueous solutions that may also contain buffers, diluents and other suitable additives such as, but not limited to, penetration enhancers, carrier compounds and other pharmaceutically acceptable carriers or excipients. Additionally, suspensions of the active compounds may be prepared as appropriate oily injection suspensions. Suitable lipophilic solvents or vehicles include fatty oils such as sesame oil, or synthetic fatty acid esters, such as ethyl oleate or triglycerides, or liposomes. Aqueous injection suspensions may contain substances that increase the viscosity of the suspension, such as sodium carboxymethyl cellulose, sorbitol, or dextran. Optionally, the suspension may also contain suitable stabilizers or agents that increase the solubility of the compounds to allow for the preparation of highly concentrated solutions.

Compositions and formulations for oral administration include powders or granules, suspensions or solutions in water or non-aqueous media, capsules, sachets or tablets. Thickeners, flavoring agents, diluents, emulsifiers, dispersing aids or binders may be desirable. Pharmaceutical preparations for oral use can be obtained by combining the active compounds with solid excipient, optionally grinding a resulting mixture, and processing the mixture of granules, after adding suitable auxiliaries, if desired, to obtain tablets or dragee cores. Suitable excipients are carbohydrate or protein fillers such as sugars, including lactose, sucrose, mannitol, or sorbitol; starch from corn, wheat, rice, potato, etc; cellulose such as methyl cellulose, hydroxypropylmethyl-cellulose, or sodium carboxymethylcellulose; and gums including arabic and tragacanth; and proteins such as gelatin and collagen. If desired, disintegrating or solubilizing agents may be added, such as the cross-linked polyvinyl pyrrolidone, agar, alginic acid or a salt thereof such as sodium alginate.

Dragee cores are provided with suitable coatings such as concentrated sugar solutions, which may also contain gum arabic, talc, polyvinylpyrrolidone, carbopol gel, polyethylene glycol, and/or titanium dioxide, lacquer solutions, and suitable organic solvents or solvent mixtures. Dyestuffs or pigments may be added to the tablets or dragee coatings for product identification or to characterize the quantity of active compound, (e.g., dosage).

Pharmaceutical preparations for oral administration include push-fit capsules made of gelatin, as well as soft, sealed capsules made of gelatin and a coating such as glycerol or sorbitol. The push-fit capsules can contain the active ingredients mixed with a filler or binders such as lactose or starches, lubricants such as talc or magnesium stearate, and, optionally, stabilizers. In soft capsules, the active compounds may be dissolved or suspended in suitable liquids, such as fatty oils, liquid paraffin, or liquid polyethylene glycol with or without stabilizers.

Compositions comprising VPA or a related form thereof formulated in a pharmaceutical acceptable carrier may be prepared, placed in an appropriate container, and labeled for treatment of an indicated condition.

Pharmaceutical formulations herein may be presented in unit dosage form, may be prepared according to conventional techniques well known in the pharmaceutical industry. Such techniques include the step of bringing into association the active ingredients with the pharmaceutical carrier(s) or excipient(s). In general the formulations are prepared by uniformly and intimately bringing into association the active ingredients with liquid carriers or finely divided solid carriers or both, and then, if necessary, shaping the product.

Dosing is dependent on severity and responsiveness of the disease state or condition to be treated, with the course of treatment lasting from several days to several months, or until a cure is effected or a diminution of the disease state of risk imposed thereby is achieved. In some embodiments, treatment is administered in one or more courses, where each course comprises one or more doses per day for several days (e.g., 1, 2, 3, 4, 5, 6) or weeks (e.g., 1, 2, or 3 weeks, etc.). In some embodiments, courses of treatment are administered sequentially (e.g., without a break between courses), while in other embodiments, a break of 1 or more days, weeks, or months is provided between courses. In some embodiments, treatment is provided on an ongoing or maintenance basis (e.g., multiple courses provided with or without breaks for an indefinite time period). Optimal dosing schedules can be calculated from measurements of drug accumulation in the body of the patient. The administering clinician can readily determine optimum dosages, dosing methodologies and repetition rates.

In some embodiments, dosage is from 0.01 μg to 100 g per kg of body weight, and may be given once or more daily, weekly, monthly or yearly. The treating clinician can estimate repetition rates for dosing based on measured residence times and concentrations of the drug in bodily fluids or tissues.

In some embodiments, the compounds disclosed herein (e.g., VPA or related forms thereof) are combined or used in combination with other agents useful in the treatment, prevention, or symtom redcuion realted to MI. Or, by way of example only, the therapeutic effectiveness of one of the compounds described herein may be enhanced by administration of an adjuvant (e.g., by itself the adjuvant may only have minimal therapeutic benefit, but in combination with another therapeutic agent, the overall therapeutic benefit to the patient is enhanced). Such other agents, adjuvants, or drugs, may be administered, by a route and in an amount commonly used therefor, simultaneously or sequentially with a compound as disclosed herein. When a compound as disclosed herein is used contemporaneously with one or more other drugs, a pharmaceutical composition containing such other drugs in addition to the compound disclosed herein may be utilized, but is not required. In some embodiments, co-administered agents are co-formulated into a single pharmaceutical compositon for administration to a subject. In some embodiments, co-administered agents are seperately formulated for individual (simultaneous or separate) administration.

In some embodiments, VPA or a related form is co-administered with an additional agent to reduce the risk of MI. Suitable additional agents for co-administration to a subject at risk (e.g., increased risk relative to the general population) of MI (e.g., sufering from congestive heart failure, sufering from atherosclerosis, sufering from myocardial ischemia, having elevated cholesterols, with a family history of heart disease, overwieght, etc.) include aspirin, a statin, clopidogrel, an anticoagulant, a beta-blocker, an ACE inhibitor, etc. Exemplary statins for use in embodiments herein include, for example, atorvastatin, fluvastatin, lovastatin, pitavastatin, pravastatin, rosuvastatin and simvastatin. Exemplary anticoagulants for use in embodiments herein include, for example, coumarins and indandiones (e.g., Warfarin, acenocoumarol, phenprocoumon, atromentin, phenindione, etc.), factor Xa inhibitors (e.g., idraparinux, fondaparinux, rivaroxaban, apixaban, edoxaban, betrixaban, darexaban, letaxaban, eribaxaban, etc.), heparins, thrombin inhibitors (e.g., hirudin, lepirudin, bivalirudin, argatroban, dabigatran, ximelagatra, etc.), etc. Exemplary beta-blockers for use in embodiments herein include, for example, aprenolol, carteolol, levobunolol, mepindolol, metipranolol, nadolol, oxprenolol, timolol, sotalol, esmolol, cateolol, propranolol, betaxolol, penbutolol, metoprolol, acebutolol, atenolol, metoprolol, pindolol, bisoprolol, nebivolol, amosulalol, landiolol, and tilisolol. Exemplary ACE inhibitors for use in embodiments herein include, for example, benazepril, captopril, enalapril fosinopril, Lisinopril, moexipril, perindopril, quinapril, Ramipril, trandolapril, etc.

In some embodiments, VPA or a related form is co-administered with an additional agent to reduce tissue damage resulting from MI and/or reperfusion. Suitable additional agents for co-administration to a subject suffering from and/or having suffered (e.g., within 1 day, 12 hours, 6 hours, 4 hours, 3 hours, 2 hours, 1 hour, 30 minutes, 15 minutes, 10 minutes, 5 minutes, 1 minute, or ranges therebetween) include, for example, aspirin, a thrombolytic agent, an antiplatelet agent, a blood thinner, a pain reliever, nitroglycerin, a beta blocker, and an ACE inhibitor. Exemplary thrombolytic agents for use in embodiments herein include, for example, anistreplase, reteplase, streptokinase, kabikinase, recombinant tissue plasminogen activators (rtPAs), tenecteplase, rokinase, urokinase, staphylokinase, etc. Exemplary antiplatelet agents for use in embodiments herein include, for example, irreversible cyclooxygenase inhibitors (e.g., aspirin, triflusal, etc.), adenosine diphosphate (ADP) receptor inhibitors (e.g., clopidogrel, prasugrel ticagrelor, ticlopidine, phosphodiesterase inhibitors, cilostazol, etc.), protease-activated receptor-1 (par-1) antagonists (e.g., vorapaxar, etc.), glycoprotein iib/iiia inhibitors (e.g., abciximab, eptifibatide, tirofiban, etc.), adenosine reuptake inhibitors (e.g., dipyridamole, etc.), thromboxane inhibitors (e.g., thromboxane synthase inhibitors, thromboxane receptor antagonists, etc.), etc. Exemplary blood thinners for use in embodiments herein include, for example, warfarin, heparin, dalteparin, bivalirudin, argatroban, lepirudin, heparin sodium, heparin/dextrose, etc. Exemplary pain relievers for use in embodiments herein include, for example, acetaminophen. Exemplary beta-blockers for use in embodiments herein include, for example, aprenolol, carteolol, levobunolol, mepindolol, metipranolol, nadolol, oxprenolol, timolol, sotalol, esmolol, cateolol, propranolol, betaxolol, penbutolol, metoprolol, acebutolol, atenolol, metoprolol, pindolol, bisoprolol, nebivolol, amosulalol, landiolol, and tilisolol. Exemplary ACE inhibitors for use in embodiments herein include, for example, benazepril, captopril, enalapril fosinopril, Lisinopril, moexipril, perindopril, quinapril, Ramipril, trandolapril, etc.

Thus, some embodiments provide methods for treating, preventing, and/or reducing damage resulting from MI, in a human or animal subject in need of such treatment, comprising administering to said subject an amount of VPA or a related form thereof, in combination with at least one additional agent that is also useful in the treatment, prevention, symptom reduction, and/or damage receptions, that is known in the art. Some embodiments provide therapeutic compositions comprising at least one compound disclosed herein in combination with one or more additional agents. Some embodiments provide combinations of multiple therapeutic compositions comprising at least one compound disclosed herein in combination with one or more additional agents.

Embodiments provided herein also include kits for use in the instant methods. Kits comprise VPA, containers or packaging, labels, instructions for use, devices for administration, agent(s) co co-administration, etc. The kit may further comprise a description of selecting an individual suitable treatment. Instructions supplied in the kits are typically written instructions on a label or package insert (e.g., a paper insert included with the kit), but machine-readable instructions (e.g., instructions carried on a magnetic or optical storage disk) are also contemplated. In some embodiments, the kit is a package containing a sealed container comprising any one of the preparations described herein, together with instructions for use. The kit can also include a diluent container containing a pharmaceutically acceptable diluent. The kit can further comprise instructions for mixing the preparation and the diluent. The diluent can be any pharmaceutically acceptable diluent. Well known diluents include 5% dextrose solution and physiological saline solution. The container can be an infusion bag, a sealed bottle, a vial, a vial with a septum, an ampoule, an ampoule with a septum, an infusion bag, or a syringe. The containers can optionally include indicia indicating that the containers have been autoclaved or otherwise subjected to sterilization techniques. The kit can include instructions for administering the various materials contained in the containers to subjects.

Embodiments herein also relate to methods of treatment with VPA. Methods are provided for treating a subject in need of such treatment with an effective amount of VPA or a related form thereof. For example, some subjects have myocardial ischemia, artherosclerosis, coronary heart disease, high blood pressure, high blood cholesterol levels, high triglyceride levels, diabetes, etc. Some subjects smoke tobacco, are over 45 years of age (e.g. over 50, over 55, over 60, etc.), are obese, have family histories of heart attack, have a history of autoimmune disease, lack physical activity in their lifestyle, etc. In some embodiments, subjects do not exhibit or suffer from conditions or diseases such as epilepsy, mania, bipolar disorder, migraine headaches, a seizure disorder, etc. The method involves administering to the subject an effective amount of a VPA or a related form thereof in any one of the pharmaceutical preparations described above, detailed herein, and/or set forth in the claims. The subject can be any subject in need of such treatment.

In some embodiments, provided herein are methods of treatment comprising: administering a pharmaceutically effective amount of VPA or a related form thereof, alone or in combination with another agent, to a subject at risk (e.g., increased risk) of suffering a heart attack (MI). In some embodiments, the administration reduces the risk of heart attack.

In some embodiments, provided herein are methods of treatment comprising: administering a pharmaceutically effective amount of VPA or a related form thereof, alone or in combination with another agent, to a subject currently suffering a heart attack (MI). In some embodiments, provided herein are methods of treatment comprising: administering a pharmaceutically effective amount of VPA or a related form thereof, alone or in combination with another agent, to a subject that has recently suffered a heart attack (MI). In some embodiments, the administration reduces tissue damage caused by the heart attack (MI) or reperfusion that follows.

In some embodiments, the methods provided comprise testing a subject for a disease or condition (e.g., that places the subject at increased risk for MI) followed by administering VPA or a related form thereof, alone or in combination with other agents. In some embodiments, methods comprise administering to a subject a VPA or a related form thereof, alone or in combination with other agents, followed by testing the subject for a disease or a condition (e.g., heart attack (e.g., tested by EKG)). In some embodiments, methods comprise testing a subject for a disease or condition (e.g., by EKG) followed by administering VPA or a related form thereof, alone or in combination with other agents, followed by a second round of testing for a disease or condition (e.g., to monitor the effect of the treatment). In some embodiments, methods comprise testing a subject for a disease or condition followed by administering VPA or a related form thereof, alone or in combination with other agents, followed by a second round of testing for a disease or condition and a second administration of VPA or a related form thereof, alone or in combination with other agents, with this second administration being modified in dose, duration, frequency, or administration route in a manner dependent upon the results of the prior testing. In some embodiments, a subject is tested to assess the presence, the absence, or the level of a disease, e.g., by assaying or measuring a biomarker, a metabolite, a physical symptom, an indication, etc. to determine the risk of or the presence of the disease and thereafter the subject is treated with VPA or a related form thereof based on the outcome of the test. In some embodiments, testing comprises performing an electrocardiogram (EKG or ECG) or other electrophysiologic monitoring (e.g., intracardiac monitoring). In some embodiments, a patient is tested, treated, and then tested again to monitor the response to therapy. In some embodiments, cycles of testing and treatment may occur without limitation to the pattern of testing and treating (e.g., test/treat, test/treat/test, test/treat/test/treat, test/treat/test/treat/test, test/treat/treat/test/treat/treat, etc.), the periodicity, or the duration of the interval between each testing and treatment phase.

### EXPERIMENTAL

### Example 1

### VPA Treatment Protects Heart Function after MI in Rats

To determine the possible protective effect of VPA after MI, MI rats were generated by ischemia reperfusion and VPA was administrated to the animals. 24 h post coronary ligation, a MI size of 33.59±3.54% of the left ventricle was induced in rats. Intra-peritoneal injection of 250 mg/kg VPA was conducted immediately after and 12 h after coronary ligation. Heart functional assays, MI assessment, and physiological assays were performed 24 h after VPA administration.

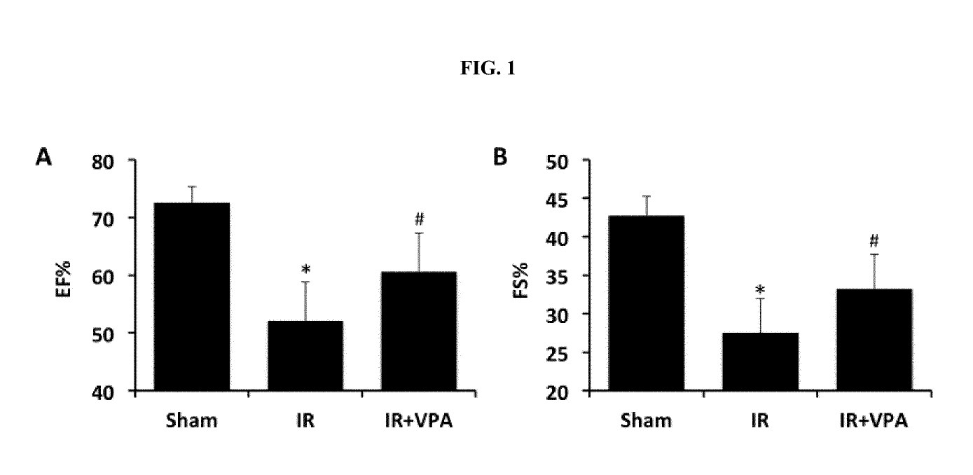

The effect of VPA on heart function was measured with echocardiography analysis of left ventricle ejection fraction (EF) and fractional shortening (FS). Left ventricular internal diameter end diastole (LVIDd) and end systole (LVIDs) were measured perpendicularly to the long axis of the ventricle. EF and FS were calculated according to LVIDd and LVIDs. IR rats showed 52.12±6.69% of EF and 27.51±4.47% of FS, whereas VPA treatment improved the EF to 60.60±6.18% and FS to 33.21±4.46%, respectively (FIG. 1). These data demonstrate that VPA treatment within 12 h of IR injury rescued roughly 50% of the functional loss caused by acute IR injury.

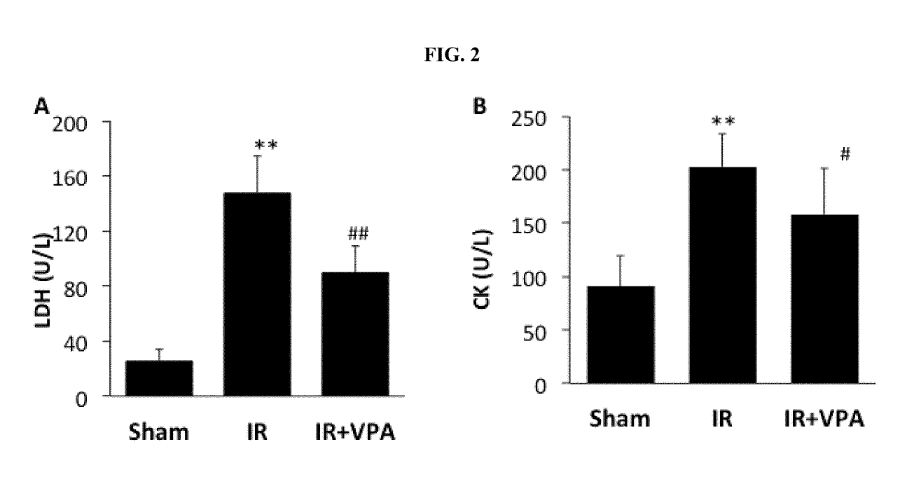

Experiments were conducted during development of embodiments herein to examine the infarct size of the animals with and without VPA treatment 24 h after injury. Myocardial infarct size was determined by triphenyltetrazolium chloride (TTC) staining. The hearts were frozen rapidly and sliced into five 2 mm transverse sections. The sections were incubated at 37° C. with 1% TTC in phosphate buffer (pH 7.4) for 10 min, fixed in 10% formaldehyde solution, photographed and calculated using ImageJ software. The infarct size was expressed as a percentage of infarct volume versus left ventricle volume. Compared to saline controls, VPA treatment significantly reduced the infarction size to 19.14±2.89% of the left ventricle (FIG. 2). Two shots of VPA within 24 h of IR injury reduced infarct size by around 45%.

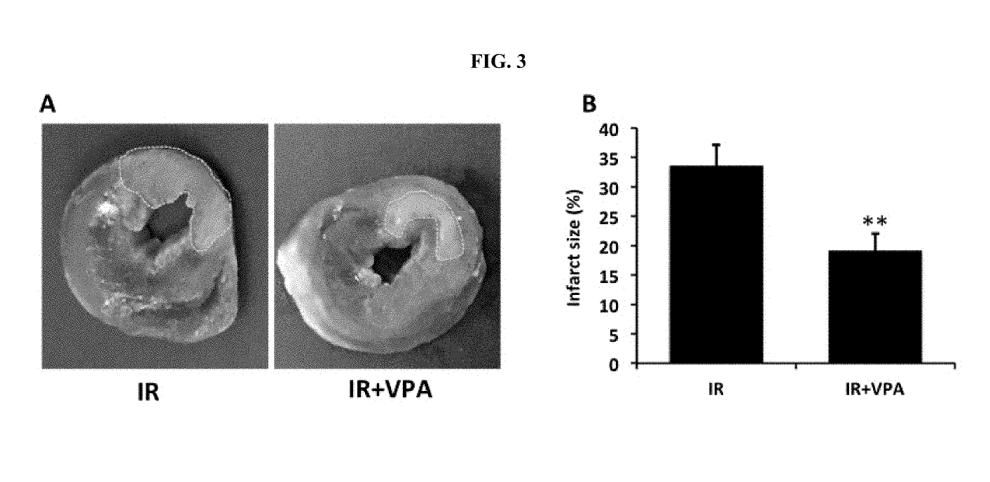

VPA mediated reduction of MI injury was further measured by plasma biochemical assay. Plasma LDH and creatine kinase (CK) levels were major biomarkers for MI injury and measured 24 h after VPA treatment using a kit (Sigma). In the IR rats without VPA treatment, high levels of LDH leakage (135.46±33.76 U/L) and CK leakage (202.88±30.63 U/L) were observed (FIG. 3). In contrast, VPA treatment significantly reduced the IR-induced release of LDH (63.07±23.83 U/L) and CK (158.31±43.53 U/L) in IR rats. In addition, long-term (4-week) treatment of VPA was performed to IR rats. Compared to IR control animals, which showed worsened EF and FS during the course, oral administration of VPA (250 mg/kg) twice daily for 4 weeks protected the MI hearts from ventricular remodeling and weakening of the pumping function.

### Example 2

### VPA Activates Foxm1 Mediated Feed-Forward Regulatory Circuitry to Open the Chromatin for Specific Gene Activation and Protect Cardiomyocytes (CMs) from Ischemia

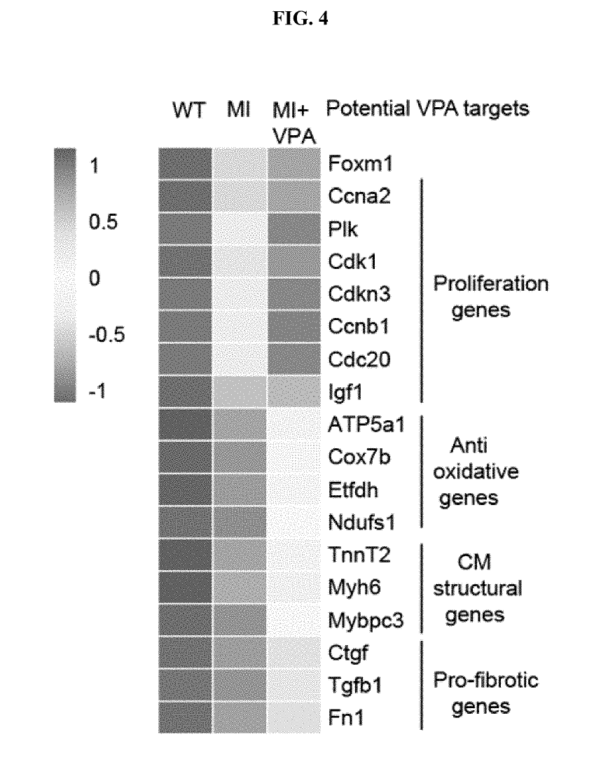

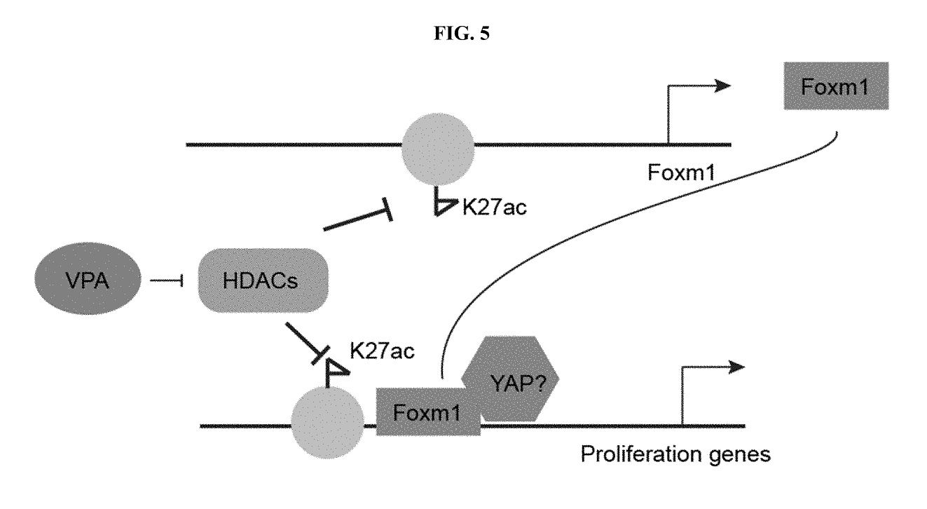

To explore the underlying mechanism of VPA-mediated heart protection, RNA-seq experiments were performed using the whole hearts one day after IR with and without VPA administration (FIG. 4). Among the genes regulated by VPA, it was detected that Foxm1 expression doubled in IR group compared to no IR surgery group. Foxm1 expression was 8-fold higher in IR+VPA group than no IR group. Recent studies indicate that neonatal CMs proliferate much more potently than adult CMs and Foxm1 appears to be a key factor in regulating neonatal CM proliferation by controlling Igf1 and cell cycle genes (Ref. 44; incorporated by reference in its entirety). Among the 180 genes mostly up-regulated by VPA treatment, 92 of them were Foxm1 targets as reported by chip-seq experiments (Ref. 45; incorporated by reference in its entirety). These genes are mostly proliferation related genes. Other potential targets include anti-oxidative, pro-fibrotic, and CM structural genes. Foxm1 has also been reported to work with YAP1 to activate proliferation genes (Ref. 46; incorporated by reference in its entirety). These experiments conducted during development of embodiments herein indicate that Foxm1 is the major mediator of VPA to forge a feed-forward regulatory circuitry to open the chromatin for specific gene activation and protect CMs from ischemia (FIG. 5).

### Example 3

8-month old WHHL rabbits are used and Osmotic pumps (Alzet Model 2ML4; Durect Corporation, Cupertino, Calif.) are placed into the subcutaneous space of ketamine/medetomidine-anesthetized rabbits through a small incision on the back of the neck that was closed with surgical sutures (Ref. 25; incorporated by reference in its entirety). 200 ng/min/kg of Ang II isinfused to the animals for three weeks. The death of the animals with VPA injection (125 mg/kg/day) and without VPA injection is monitored every six hours and histological features of fresh MI, including myocardial eosinophilic degeneration, disappearance of striation, coagulation necrosis, edema, neutrophil infiltration, and hemorrhage are examined. Abnormalities in other organs, such as liver, kidneys, brain, adrenals, stomach, and intestines are examined as well.

### Example 4

WT NZW rabbits are anaesthetized with 2% isoflurane inhalation using an isoflurane delivery system. AMI/R is performed by temporary occlusion of the left descending coronary artery (LAD) using 6-0 silk suture and a PE10 tube placement around LAD. After 60 min of ischemia, the PE10 tube is removed, allowing the myocardium to be re-perfused. Animals are randomly assigned to three experimental groups: (i) Sham group (undergoing the same surgical procedure except for occlusion); (ii) AMI/R group; and (iii) AMUR+VPA (120 mg/kg) group. Rabbits in AMI/R+VPA group are injected intraperitoneally with VPA immediately before LAD occlusion. The rabbits in Sham and AMI/R groups are administered with a corresponding dose of the saline. Another dose was administered at 12 h after surgery. Echocardiographic, infarct size assessment and plasma biochemical assay are performed 24 h after I/R and VAP treatment.

### REFERENCES

The following references, some of which are cited above by number, are herein incorporated by reference in their entireties.
1. Writing Group M, et al. Circulation 133(4):447-454.
2. Chong & Sullivan. (2007) New uses for old drugs. Nature 448(7154):645-646.
3. Allison (2012) NCATS launches drug repurposing program. Nat Biotechnol 30(7):571-572.
4. Mullard (2012) Drug repurposing programmes get lift off. Nat Rev Drug Discov 11(7):505-506.
5. Nosengo (2016) Can you teach old drugs new tricks? Nature 534(7607):314-316.
6. Berry et al. (2008) Histone deacetylase inhibition in the treatment of heart disease. Expert Opin Drug Saf 7(1):53-67.
7. Haberland et al. (2009) The many roles of histone deacetylases in development and physiology: implications for disease and therapy. Nat Rev Genet 10(1):32-42.
8. McKinsey (2012) Therapeutic potential for HDAC inhibitors in the heart. Annu Rev Pharmacol Toxicol 52:303-319.
9. Dokmanovic et al. (2007) Histone deacetylase inhibitors: overview and perspectives. Mol Cancer Res 5(10):981-989.
10. New M, Olzscha H, & La Thangue NB (2012) HDAC inhibitor-based therapies: can we interpret the code? Mol Oncol 6(6):637-656.
11. Papavassiliou & Papavassiliou (2014) Histone deacetylases inhibitors: conjugation to other anti-tumour pharmacophores provides novel tools for cancer treatment. Expert Opin Investig Drugs 23(3):291-294.
12. Yang et al. (2016) The emerging role of extracellular vesicle-derived miRNAs: implication in cancer progression and stem cell related diseases. J Clin Epigenet 2(1).
13. Zhao et al. (2007) Inhibition of histone deacetylases triggers pharmacologic preconditioning effects against myocardial ischemic injury. Cardiovasc Res 76(3):473-481.
14. Eom & Kook (2014) Posttranslational modifications of histone deacetylases: implications for cardiovascular diseases. Pharmacol Ther 143(2):168-180.
15. Xie et al. (2014) Histone deacetylase inhibition blunts ischemia/reperfusion injury by inducing cardiomyocyte autophagy. Circulation 129(10):1139-1151.
16. Goey et al. (2016) Pharmacogenomics and histone deacetylase inhibitors. Pharmacogenomics 17(16):1807-1815.
17. Olesen et al. (2011) Valproate attenuates the risk of myocardial infarction in patients with epilepsy: a nationwide cohort study. Pharmacoepidemiol Drug Saf 20(2):146-153.
18. Dregan et al. (2014) Is sodium valproate, an HDAC inhibitor, associated with reduced risk of stroke and myocardial infarction? A nested case-control study. Pharmacoepidemiol Drug Saf 23(7):759-967.
19. Halaweish et al. (2015) Addition of low-dose valproic acid to saline resuscitation provides neuroprotection and improves long-term outcomes in a large animal model of combined traumatic brain injury and hemorrhagic shock. J Trauma Acute Care Surg 79(6):911-919; discussion 919.
20. Bambakidis et al. (2016) Resuscitation with Valproic Acid Alters Inflammatory Genes in a Porcine Model of Combined Traumatic Brain Injury and Hemorrhagic Shock. J Neurotrauma 33(16):1514-1521.
21. Fan & Watanabe (2003) Transgenic rabbits as therapeutic protein bioreactors and human disease models. Pharmacol Ther 99(3):261-282.
22. Fan et al. (2015) Rabbit models for the study of human atherosclerosis: from pathophysiological mechanisms to translational medicine. Pharmacol Ther 146:104-119.
23. Watanabe (1980) Serial inbreeding of rabbits with hereditary hyperlipidemia (WHHL-rabbit). Atherosclerosis 36(2):261-268.
24. Shiomi et al. (2003) Development of an animal model for spontaneous myocardial infarction (WHHLMI rabbit). Arterioscler Thromb Vasc Biol 23(7):1239-1244.
25. Li et al. (2016) Angiotensin II Destabilizes Coronary Plaques in Watanabe Heritable Hyperlipidemic Rabbits. Arterioscler Thromb Vasc Biol 36(5):810-816.
26. Guan et al. (2014) Roles of deletion of Aridl a, a tumor suppressor, in mouse ovarian tumorigenesis. J Natl Cancer Inst 106(7):1-4.
27. He et al. (2014) BAF200 is required for heart morphogenesis and coronary artery development. PLoS One 9(10):e109493.
28. Helming et al. (2014) ARID1B is a specific vulnerability in ARID1A-mutant cancers. Nat Med 20(3):251-254.
29. Li et al. (2014) Rosa26-targeted swine models for stable gene over-expression and Cre-mediated lineage tracing. Cell research 24(4):501-504.
30. Wu et al. (2014) Baf250a orchestrates an epigenetic pathway to repress the Nkx2.5-directed contractile cardiomyocyte program in the sinoatrial node. Cell research 24(10):1201-1213.
31. Kim et al. (2015) ARID1A Is Essential for Endometrial Function during Early Pregnancy. PLoS Genet 11(9):e1005537.
32. Lei I, Bu L, & Wang Z (2015) Derivation of cardiac progenitor cells from embryonic stem cells. J Vis Exp 95:52047.
33. Lei et al. (2015) BAF250a Protein Regulates Nucleosome Occupancy and Histone Modifications in Priming Embryonic Stem Cell Differentiation. J Biol Chem 290(31):19343-19352.
34. Liu et al. (2015) Porous nanofibrous poly(l-lactic acid) scaffolds supporting cardiovascular progenitor cells for cardiac tissue engineering. Acta Biomaterialia 26:105-114.
35. Xie et al. (2015) Ductile electroactive biodegradable hyperbranched polylactide copolymers enhancing myoblast differentiation. Biomaterials 71:158-167.
36. Tian et al. (2015) Heart regeneration with embryonic cardiac progenitor cells and cardiac tissue engineering. J Stem Cell and Transplantation Biology 1(1):104.
37. Lee et al. (2016) BAF180 regulates cellular senescence and hematopoietic stem cell homeostasis through p21. Oncotarget 7(15):19134-19146.
38. Wang et al. (2016) Deletion of Aridla in Reproductive Tract Mesenchymal Cells Reduces Fertility in Female Mice. Biol Reprod 94(4):93.
39. Yang et al. (2016) Identification and characterization of rabbit Rosa26 for gene knock-in and stable reporter gene expression. Scientific Reports 6:25161.
40. Zhai et al. (2016) Aridla inactivation in an Apc- and Pten-defective mouse ovarian cancer model enhances epithelial differentiation and prolongs survival. J Pathol 238(1):21-30.
41. Liu et al. (2016) Improving cardiac reprogramming for heart regeneration. Curr Opin Organ Transplant 21(6):588-594.
42. Sun et al. (2016) Suppression of the SWI/SNF Component Aridla Promotes Mammalian Regeneration. Cell Stem Cell 18(4):456-466.
43. Liu et al. (2016) Targeting Mll 1 H3K4 methyltransferase activity to guide cardiac lineage specific reprogramming of fibroblasts. Cell Discovery 2:16036.
44. Sengupta et al. (2013) FoxO1 and FoxM1 transcription factors have antagonistic functions in neonatal cardiomyocyte cell-cycle withdrawal and IGF1 gene regulation. Circ Res 112(2):267-277.
45. Chen et al. (2013) The forkhead transcription factor FOXM1 controls cell cycle-dependent gene expression through an atypical chromatin binding mechanism. Mol Cell Biol 33(2):227-236.
46. Eisinger-Mathason et al. (2015) Deregulation of the Hippo pathway in soft-tissue sarcoma promotes FOXM1 expression and tumorigenesis. Proc Natl Acad Sci USA 112(26):E3402-3411.

1. A method of reducing the risk of myocardial infarction (MI) and/or reducing tissue damage caused by MI in a subject comprising administering to the subject an effective dose of valproic acid or a related form thereof, wherein the subject does not suffer from epilepsy, mania, bipolar disorder, migrane headaches, or a seizure disorder.

2. The method of claim 1, wherein the subject is at increased risk of MI relative to the general population, and is administered valproic acid or a related form thereof before an MI event occurs.

3. The method of claim 2, wherein the subject suffers from myocardial ischemia, congenital heart disease, and/or atherosclerosis.

4. The method of claim 2, wherein the subject is administered a related from of valproic acid selected from the group consisting of valproate, sodium valproate, and divalproex.

5. The method of claim 1, wherein the valproic acid or related form thereof is administered orally or by injection.

6. The method of claim 1, wherein the valproic acid or related form thereof is co-administered with one or more additional prophylactic agents.

7. The method of claim 6, wherein the one or more additional prophylactic agents are selected from the group consisting of aspirin, a statin, clopidogrel, an anticoagulant, a beta-blocker, and an Ace inhibitor.

8. The method of claim 7, wherein the valproic acid or related form thereof and the one or more additional prophylactic agents are co-formulated.

9. The method of claim 7, wherein the valproic acid or related form thereof and the one or more additional prophylactic agents are contained in separate pharmaceutical compositions.

10. (canceled)

11. The method of claim 1, wherein the administration of valproic acid or a related form thereof reduces tissue damage caused by the MI event itself.

12. The method of claim 1, wherein the administration of valproic acid or a related form thereof reduces tissue damage caused by reperfusion.

13.-15. (canceled)

16. The method of claim 1, wherein the subject is administered valproic acid or a related form thereof during an MI event.

17. The method of claim 1, wherein the subject is administered valproic acid or a related form thereof after an MI event.

18.-20. (canceled)

21. The method of claim 5, wherein the valproic acid or related form thereof is injected intraperitoneally.

22. A prophylactic pharmaceutical composition for the prevention of myocardial infarction comprising co-formulated: (i) valproic acid or a related form thereof, and (ii) aspirin, a statin, clopidogrel, an anticoagulant, a beta-blocker, and an ACE inhibitor.

23. A kit for the prophylactic prevention of myocardial infarction comprising separately-formulated: (i) valproic acid or a related form thereof, and (ii) aspirin, a statin, clopidogrel, an anticoagulant, a beta-blocker, and an ACE inhibitor.

24. A pharmaceutical composition for treatment and/or reduction of tissue damage from myocardial infarction comprising co-formulated: (i) valproic acid or a related form thereof, and (ii) aspirin, a thrombolytic agent, an antiplatelet agent, a blood thinner, a pain reliever, nitroglycerin, a beta blocker, and an ACE inhibitor.

25. A kit for the treatment of myocardial infarction and/or reduction of tissue damage from myocardial infarction comprising separately-formulated: (i) valproic acid or a related form thereof, and (ii) aspirin, a statin, clopidogrel, an anticoagulant, a beta-blocker, and an ACE inhibitor.
